SEQUENCE LISTING (108 sequences)

SEQ ID NO: 1 - DNA, 82 bp, Artificial Sequence
  Amplicon
tgtgagtgaaatgccttctagtagtgaaccgtcctcgggagccgactatgactactcagaagagtatgataacgaaccacaa

SEQ ID NO: 2 - DNA, 73 bp, Artificial Sequence
  Amplicon
cagatggacctagtacccactgagatttccacgccgaaggacagcgatgggaaaaatgcccttaaatcatagg

SEQ ID NO: 3 - DNA, 78 bp, Artificial Sequence
  Amplicon
gacgaagacagtccctggatcaccgacagcacagacagaatccctgctaccagagaccaagacacattccaccccagt

SEQ ID NO: 4 - DNA, 71 bp, Artificial Sequence
  Amplicon
tcttgctggctacgcctcttctgtccctgttagacgtcctccgtccatatcagaactgtgccacaatgcag

SEQ ID NO: 5 - DNA, 66 bp, Artificial Sequence
  Amplicon
aaagcctcagtcagccaagtggaggcggacttgaaaatgctcaggactgctgtggacagtttggtt

SEQ ID NO: 6 - DNA, 62 bp, Artificial Sequence
  Amplicon
ggccgagatctacaaaaacggccccgtggagggagctttctctgtgtattcggacttcctgc

SEQ ID NO: 7 - DNA, 73 bp, Artificial Sequence
  Amplicon
cacaatggcggctctgaagagttggctgtcgcgcagcgtaacttcattcttcaggtacagacagtgtttgtgt

SEQ ID NO: 8 - DNA, 81 bp, Artificial Sequence
  Amplicon
cggttatgtcatgccagatacacacctcaaaggtactccctcctcccgggaaggcaccctttcttcagtgggtctcagttc

SEQ ID NO: 9 - DNA, 74 bp, Artificial Sequence
  Amplicon
cacgggacattcaccacatcgactactataaaaagacaaccaacggccgactgcctgtgaagtggatggcaccc

SEQ ID NO: 10 - DNA, 68 bp, Artificial Sequence
  Amplicon
tcagcagcaagggcatcatggaggaggatgaggcctgcgggcgccagtacacgctcaagaaaaccacc

SEQ ID NO: 11 - DNA, 73 bp, Artificial Sequence
  Amplicon
cccactcagtagccaagtcacaatgtttggaaaacagcccgtttacttgagcaagactgataccacctgcgtg

SEQ ID NO: 12 - DNA, 71 bp, Artificial Sequence
  Amplicon
ccggaaaggccaagacaaaggcggtttcccgctcgcagagagccggcttgcagttcccagtgggccgtatt

SEQ ID NO: 13 - DNA, 84 bp, Artificial Sequence
  Amplicon
aggctgctggaggtcatctccgtgtgtgattgccccagaggccgtttcttggccgccatctgccaagactgtggccgcaggaag

SEQ ID NO: 14 - DNA, 83 bp, Artificial Sequence
  Amplicon
gcatggtagccgaagatttcacagtcaaaatcggagattttggtatgacgcgagatatctatgagacagactattaccggaaa

SEQ ID NO: 15 - DNA, 77 bp, Artificial Sequence
  Amplicon
ccgccctcacctgaagagaaacgcgctccttggcggacactgggggaggagaggaagaagcgcggctaacttattcc

SEQ ID NO: 16 - DNA, 74 bp, Artificial Sequence
  Amplicon
ccaaccctgcagactccaagcctgggaccatccgtggagacttctgcatacaagttggcaggaacattatacat

SEQ ID NO: 17 - DNA, 83 bp, Artificial Sequence
  Amplicon
cgagactctcctcatagtgaaaggtatgtgtcagccatgaccaccccggctcgtatgtcacctgtagatttccacacgccaag

SEQ ID NO: 18 - DNA, 72 bp, Artificial Sequence
  Amplicon
gggagtttccaagagatggactagtgcttggtcgggtcttggggtctggagcgtttgggaaggtggttgaag

SEQ ID NO: 19 - DNA, 69 bp, Artificial Sequence
  Amplicon
agtgggagacacctgacctttctcaagctgagattgagcagaagatcaaggagtacaatgcccagatca

SEQ ID NO: 20 - DNA, 64 bp, Artificial Sequence
  Amplicon
cctgaacatgaaggagctgaagctgctgcagaccatcgggaagggggagttcggagacgtgatg

SEQ ID NO: 21 - DNA, 77 bp, Artificial Sequence
  Amplicon
gaggcgctcaacatgaaattcaaggccgaagtgcagagcaaccggggcctgaccaaggagaacctcgtgttcctggc

SEQ ID NO: 22 - DNA, 74 bp, Artificial Sequence
  Amplicon
ccagtggtggtgatcgttcatggcagccaggacaacaatgcgacggccactgttctctgggacaatgcttttgc

SEQ ID NO: 23 - DNA, 73 bp, Artificial Sequence
  Amplicon
gatggagcaggtggctcagttcctgaaggcggctgaggactctggggtcatcaagactgacatgttccagact

SEQ ID NO: 24 - DNA, 69 bp, Artificial Sequence
  Amplicon
tcaccctctgtgacttcatcgtgccctgggacaccctgagcaccacccagaagaagagcctgaaccaca

SEQ ID NO: 25 - DNA, 71 bp, Artificial Sequence
  Amplicon
ctgctgtcttgggtgcattggagccttgccttgctgctctacctccaccatgccaagtggtcccaggctgc

SEQ ID NO: 26 - DNA, 75 bp, Artificial Sequence
  Amplicon
agaggcatccatgaacttcacacttgcgggctgcatcagcacacgctcctatcaacccaagtactgtggagtttg

SEQ ID NO: 27 - DNA, 76 bp, Artificial Sequence
  Amplicon
agactgtggagtttgatgttgttgaaggagaaaagggtgcggaggcagcaaatgttacaggtcctggtggtgttcc

SEQ ID NO: 28 - DNA, 27 bp, Artificial Sequence
  forward primer
tgtgagtgaaatgccttctagtagtga

SEQ ID NO: 29 - DNA, 23 bp, Artificial Sequence
  probe
ccgtcctcgggagccgactatga

SEQ ID NO: 30 - DNA, 27 bp, Artificial Sequence
  reverse primer
ttgtggttcgttatcatactcttctga

SEQ ID NO: 31 - DNA, 25 bp, Artificial Sequence
  forward primer
cagatggacctagtacccactgaga

SEQ ID NO: 32 - DNA, 22 bp, Artificial Sequence
  probe
ttccacgccgaaggacagcgat

SEQ ID NO: 33 - DNA, 24 bp, Artificial Sequence
  reverse primer
cctatgatttaagggcatttttcc

SEQ ID NO: 34 - DNA, 20 bp, Artificial Sequence
  forward primer
gacgaagacagtccctggat

SEQ ID NO: 35 - DNA, 20 bp, Artificial Sequence
  reverse primer
actggggtggaatgtgtctt

SEQ ID NO: 36 - DNA, 24 bp, Artificial Sequence
  probe
caccgacagcacagacagaatccc

SEQ ID NO: 37 - DNA, 20 bp, Artificial Sequence
  forward primer
tcttgctggctacgcctctt

SEQ ID NO: 38 - DNA, 28 bp, Artificial Sequence
  probe
tgtccctgttagacgtcctccgtccata

SEQ ID NO: 39 - DNA, 21 bp, Artificial Sequence
  reverse primer
ctgcattgtggcacagttctg

SEQ ID NO: 40 - DNA, 20 bp, Artificial Sequence
  forward primer
aaagcctcagtcagccaagt

SEQ ID NO: 41 - DNA, 20 bp, Artificial Sequence
  reverse primer
aaccaaactgtccacagcag

SEQ ID NO: 42 - DNA, 24 bp, Artificial Sequence
  probe
tcctgagcattttcaagtccgcct

SEQ ID NO: 43 - DNA, 20 bp, Artificial Sequence
  forward primer
ggccgagatctacaaaaacg

SEQ ID NO: 44 - DNA, 20 bp, Artificial Sequence
  reverse primer
gcaggaagtccgaatacaca

SEQ ID NO: 45 - DNA, 21 bp, Artificial Sequence
  probe
ccccgtggagggagctttctc

SEQ ID NO: 46 - DNA, 19 bp, Artificial Sequence
  forward primer
cacaatggcggctctgaag

SEQ ID NO: 47 - DNA, 26 bp, Artificial Sequence
  reverse primer
acacaaacactgtctgtacctgaaga

SEQ ID NO: 48 - DNA, 23 bp, Artificial Sequence
  probe
aagttacgctgcgcgacagccaa

SEQ ID NO: 49 - DNA, 23 bp, Artificial Sequence
  forward primer
cggttatgtcatgccagatacac

SEQ ID NO: 50 - DNA, 25 bp, Artificial Sequence
  probe
cctcaaaggtactccctcctcccgg

SEQ ID NO: 51 - DNA, 24 bp, Artificial Sequence
  reverse primer
gaactgagacccactgaagaaagg

SEQ ID NO: 52 - DNA, 20 bp, Artificial Sequence
  forward primer
cacgggacattcaccacatc

SEQ ID NO: 53 - DNA, 19 bp, Artificial Sequence
  reverse primer
gggtgccatccacttcaca

SEQ ID NO: 54 - DNA, 27 bp, Artificial Sequence
  probe
ataaaaagacaaccaacggccgactgc

SEQ ID NO: 55 - DNA, 19 bp, Artificial Sequence
  forward primer
tcagcagcaagggcatcat

SEQ ID NO: 56 - DNA, 23 bp, Artificial Sequence
  reverse primer
ggtggttttcttgagcgtgtact

SEQ ID NO: 57 - DNA, 19 bp, Artificial Sequence
  probe
cgcccgcaggcctcatcct

SEQ ID NO: 58 - DNA, 20 bp, Artificial Sequence
  forward primer
cccactcagtagccaagtca

SEQ ID NO: 59 - DNA, 27 bp, Artificial Sequence
  probe
tcaagtaaacgggctgttttccaaaca

SEQ ID NO: 60 - DNA, 20 bp, Artificial Sequence
  reverse primer
cacgcaggtggtatcagtct

SEQ ID NO: 61 - DNA, 18 bp, Artificial Sequence
  forward primer
ccggaaaggccaagacaa

SEQ ID NO: 62 - DNA, 20 bp, Artificial Sequence
  reverse primer
aatacggcccactgggaact

SEQ ID NO: 63 - DNA, 19 bp, Artificial Sequence
  probe
cccgctcgcagagagccgg

SEQ ID NO: 64 - DNA, 20 bp, Artificial Sequence
  forward primer
aggctgctggaggtcatctc

SEQ ID NO: 65 - DNA, 19 bp, Artificial Sequence
  reverse primer
cttcctgcggccacagtct

SEQ ID NO: 66 - DNA, 21 bp, Artificial Sequence
  probe
ccagaggccgtttcttggccg

SEQ ID NO: 67 - DNA, 21 bp, Artificial Sequence
  forward primer
gcatggtagccgaagatttca

SEQ ID NO: 68 - DNA, 30 bp, Artificial Sequence
  reverse primer
tttccggtaatagtctgtctcatagatatc

SEQ ID NO: 69 - DNA, 28 bp, Artificial Sequence
  probe
cgcgtcataccaaaatctccgattttga

SEQ ID NO: 70 - DNA, 19 bp, Artificial Sequence
  forward primer
ccgccctcacctgaagaga

SEQ ID NO: 71 - DNA, 22 bp, Artificial Sequence
  reverse primer
ggaataagttagccgcgcttct

SEQ ID NO: 72 - DNA, 21 bp, Artificial Sequence
  probe
cccagtgtccgccaaggagcg

SEQ ID NO: 73 - DNA, 19 bp, Artificial Sequence
  forward primer
ccaaccctgcagactccaa

SEQ ID NO: 74 - DNA, 28 bp, Artificial Sequence
  reverse primer
atgtataatgttcctgccaacttgtatg

SEQ ID NO: 75 - DNA, 25 bp, Artificial Sequence
  probe
cctgggaccatccgtggagacttct

SEQ ID NO: 76 - DNA, 27 bp, Artificial Sequence
  forward primer
cgagactctcctcatagtgaaaggtat

SEQ ID NO: 77 - DNA, 22 bp, Artificial Sequence
  reverse primer
cttggcgtgtggaaatctacag

SEQ ID NO: 78 - DNA, 24 bp, Artificial Sequence
  probe
atgaccaccccggctcgtatgtca

SEQ ID NO: 79 - DNA, 20 bp, Artificial Sequence
  forward primer
gggagtttccaagagatgga

SEQ ID NO: 80 - DNA, 23 bp, Artificial Sequence
  probe
cccaagacccgaccaagcactag

SEQ ID NO: 81 - DNA, 20 bp, Artificial Sequence
  reverse primer
cttcaaccaccttcccaaac

SEQ ID NO: 82 - DNA, 20 bp, Artificial Sequence
  forward primer
agtgggagacacctgacctt

SEQ ID NO: 83 - DNA, 20 bp, Artificial Sequence
  reverse primer
tgatctgggcattgtactcc

SEQ ID NO: 84 - DNA, 29 bp, Artificial Sequence
  probe
ttgatcttctgctcaatctcagcttgaga

SEQ ID NO: 85 - DNA, 20 bp, Artificial Sequence
  forward primer
cctgaacatgaaggagctga

SEQ ID NO: 86 - DNA, 19 bp, Artificial Sequence
  reverse primer
catcacgtctccgaactcc

SEQ ID NO: 87 - DNA, 21 bp, Artificial Sequence
  probe
tcccgatggtctgcagcagct

SEQ ID NO: 88 - DNA, 21 bp, Artificial Sequence
  forward primer
gaggcgctcaacatgaaattc

SEQ ID NO: 89 - DNA, 20 bp, Artificial Sequence
  reverse primer
gccaggaacacgaggttctc

SEQ ID NO: 90 - DNA, 22 bp, Artificial Sequence
  probe
cggttgctctgcacttcggcct

SEQ ID NO: 91 - DNA, 20 bp, Artificial Sequence
  forward primer
ccagtggtggtgatcgttca

SEQ ID NO: 92 - DNA, 21 bp, Artificial Sequence
  reverse primer
gcaaaagcattgtcccagaga

SEQ ID NO: 93 - DNA, 23 bp, Artificial Sequence
  probe
cagccaggacaacaatgcgacgg

SEQ ID NO: 94 - DNA, 20 bp, Artificial Sequence
  forward primer
gatggagcaggtggctcagt

SEQ ID NO: 95 - DNA, 25 bp, Artificial Sequence
  reverse primer
agtctggaacatgtcagtcttgatg

SEQ ID NO: 96 - DNA, 24 bp, Artificial Sequence
  probe
cccagagtcctcagccgccttcag

SEQ ID NO: 97 - DNA, 22 bp, Artificial Sequence
  forward primer
tcaccctctgtgacttcatcgt

SEQ ID NO: 98 - DNA, 22 bp, Artificial Sequence
  reverse primer
tgtggttcaggctcttcttctg

SEQ ID NO: 99 - DNA, 22 bp, Artificial Sequence
  probe
ccctgggacaccctgagcacca

SEQ ID NO: 100 - DNA, 20 bp, Artificial Sequence
  forward primer
ctgctgtcttgggtgcattg

SEQ ID NO: 101 - DNA, 25 bp, Artificial Sequence
  probe
ttgccttgctgctctacctccacca

SEQ ID NO: 102 - DNA, 18 bp, Artificial Sequence
  reverse primer
gcagcctgggaccacttg

SEQ ID NO: 103 - DNA, 22 bp, Artificial Sequence
  forward primer
agaggcatccatgaacttcaca

SEQ ID NO: 104 - DNA, 24 bp, Artificial Sequence
  reverse primer
caaactccacagtacttgggttga

SEQ ID NO: 105 - DNA, 20 bp, Artificial Sequence
  probe
cgggctgcatcagcacacgc

SEQ ID NO: 106 - DNA, 25 bp, Artificial Sequence
  forward primer
agactgtggagtttgatgttgttga

SEQ ID NO: 107 - DNA, 22 bp, Artificial Sequence
  reverse primer
ggaacaccaccaggacctgtaa

SEQ ID NO: 108 - DNA, 23 bp, Artificial Sequence
  probe
ttgctgcctccgcacccttttct

(19) United States
(12) Patent Application Publication
(10) Pub. No.: US 2008/0176229 A1
(43) Pub. Date: Jul. 24, 2008

(54) Gene Expression Markers for Response to EGFR Inhibitor Drugs

(76) Inventors: David Agus, Beverly Hills, CA (US); Steven Shak, Hillsborough, CA (US); Maureen Cronin, Los Altos, CA (US); Joffre Baker, Montara, CA (US)

Correspondence Address: HELLER EHRMAN LLP, 275 MIDDLEFIELD ROAD, MENLO PARK, CA, 94025-3506, US

(21) Appl. No.: 11/755,697

(22) Filed: May 30, 2007

Related U.S. Application Data
(62) Division of application No. 10/773,951, filed on Feb. 6, 2004, now abandoned.
(60) Provisional application No. 60/445,968, filed on Feb. 6, 2003.

(51) Int. Cl. C12Q 1/68; G01N 33/574; C40B 40/08; C12P 19/34

(52) U.S. Cl. 435/6; 435/7.23; 506/17; 435/91.2

(57) ABSTRACT

The present invention concerns prognostic markers associated with cancer. In particular, the invention concerns prognostic methods based on the molecular characterization of gene expression in paraffin-embedded, fixed samples of cancer tissue, which allow a physician to predict whether a patient is likely to respond well to treatment with an EGFR inhibitor.

The present application is a divisional application under 35 U.S.C. 121 of U.S. application Ser. No. 10/773,951 filed Feb. 6, 2004, which claims the benefit under 35 U.S.C. 119(e) of U.S. Provisional Application Ser. No. 60/445,968, filed Feb. 6, 2003, the entire disclosures which are hereby incorporated by reference.

### BACKGROUND OF THE INVENTION

1. Field of the Invention

The present invention concerns gene expression profiling of tissue samples obtained from patients who are candidates for treatment with a therapeutic EGFR inhibitor. More specifically, the invention provides methods based on the molecular characterization of gene expression in paraffin-embedded, fixed cancer tissue samples, which allow a physician to predict whether a patient is likely to respond well to treatment with an EGFR inhibitor.

2. Description of the Related Art

Oncologists have a number of treatment options available to them, including different combinations of chemotherapeutic drugs that are characterized as “standard of care,” and a number of drugs that do not carry a label claim for particular cancer, but for which there is evidence of efficacy in that cancer. Best likelihood of good treatment outcome requires that patients be assigned to optimal available cancer treatment, and that this assignment be made as quickly as possible following diagnosis.

Currently, diagnostic tests used in clinical practice are single analyte, and therefore do not capture the potential value of knowing relationships between dozens of different markers. Moreover, diagnostic tests are frequently not quantitative, relying on immunohistochemistry. This method often yields different results in different laboratories, in part because the reagents are not standardized, and in part because the interpretations are subjective and cannot be easily quantified. RNA-based tests have not often been used because of the problem of RNA degradation over time and the fact that it is difficult to obtain fresh tissue samples from patients for analysis. Fixed paraffin-embedded tissue is more readily available. Fixed tissue has been routinely used for non-quantitative detection of RNA, by in situ hybridization. However, recently methods have been established to quantify RNA in fixed tissue, using RT-PCR. This technology platform can also form the basis for multi-analyte assays

Recently, several groups have published studies concerning the classification of various cancer types by microarray gene expression analysis (see, e.g. Golub et al. Science 286:531-537 (1999); Bhattachajae et al., Proc. Natl. Acad. Sci. USA 98:13790-13795 (2001); Chen-Hsiang et al., Bioinformatics 17 (Suppl. 1):S316-S322 (2001); Ramaswamy et al., Proc. Natl. Acad. Sci. USA 98:15149-15154 (2001)). Certain classifications of human breast cancers based on gene expression patterns have also been reported (Martin et al, Cancer Res. 60:2232-2238 (2000); West et al. Proc. Natl. Acad Sci. USA 98:11462-11467 (2001); Sorlie et al, Proc. Natl. Acad. Sci. USA 98:10869-10874 (2001); Yan et al, Cancer Res. 61:8375-8380 (2001)). However, these studies mostly focus on improving and refining the already established classification of various types of cancer, including breast cancer, and generally do not link the findings to treatment strategies in order to improve the clinical outcome of cancer therapy.

Although modern molecular biology and biochemistry have revealed hundreds of genes whose activities influence the behavior of tumor cells, the state of their differentiation, and their sensitivity or resistance to certain therapeutic drugs, with a few exceptions, the status of these genes has not been exploited for the purpose of routinely making clinical decisions about drug treatments. One notable exception is the use of estrogen receptor (ER) protein expression in breast carcinomas to select patients to treatment with anti-estrogen drugs, such as tamoxifen. Another exceptional example is the use of ErbB2 (Her2) protein expression in breast carcinomas to select patients with the Her2 antagonist drug Herceptin® (Genentech, Inc., South San Francisco, Calif.).

Despite recent advances, a major challenge in cancer treatment remains to target specific treatment regimens to pathogenically distinct tumor types, and ultimately personalize tumor treatment in order to optimize outcome. Hence, a need exists for tests that simultaneously provide predictive information about patient responses to the variety of treatment options.

### SUMMARY OF THE INVENTION

The present invention is based on findings of a Phase II clinical study of gene expression in tissue samples obtained from human patients with non-small cell lung cancer (NSCLC) who responded or did not respond to treatment with EGFR inhibitors.

In one embodiment, the invention concerns a method for predicting the likelihood that a patient who is a candidate for treatment with an EGFR inhibitor will respond to such treatment, comprising determining the expression level of one or more prognostic RNA transcripts or their expression products in a cancer tissue sample obtained from the patient, wherein the prognostic transcript is the transcript of one or more genes selected from the group consisting of: STAT5A, STAT5B, WISP1, CKAP4, FGFR1, cdc25A, RASSF1, G-Catenin, H2AFZ, NME1, NRG1, BC12, TAGLN, YB-1, Src, IGF1R, CD44, DIABLO, TIMP2, AREG, PDGFRa, CTSB, Hepsin, ErbB3, MTA1, Gus, and VEGF, wherein (a) over-expression of the transcript of one or more of STAT5A, STAT5B, WISP1, CKAP4, FGFR1, cdc25A, RASSF1, G-Catenin, H2AFZ, NME1, NRG1, BC12, TAGLN, YB1, Src, IGF1R, CD44, DIABLO, TIMP2, AREG, PDGFRa, and CTSB, or the corresponding expression product, indicates that the patient is not likely to respond well to the treatment, and (b) over-expression of the transcript of one or more of Hepsin, ErbB3, MTA, Gus, and VEGF, or the corresponding expression product, indicates that the patient is likely to respond well to the treatment.

The tissue sample preferably is a fixed, paraffin-embedded tissue. Tissue can be obtained by a variety of methods, including fine needle, aspiration, bronchial lavage, or transbronchial biopsy.

In a specific embodiment, the expression level of the prognostic RNA transcript or transcripts is determined by RT-PCR. In this case, and when the tissue sample is fixed, and paraffin-embedded, the RT-PCR amplicons (defined as the polynucleotide sequence spanned by the PCR primers) should preferably be less than 100 bases in length. In other embodiments, the levels of the expression product of the prognostic RNA transcripts are determined by other methods known in the art, such as immunohistochemistry, or proteomics technology. The assays for measuring the prognostic RNA transcripts or their expression products may be available in a kit format.

In another aspect, the invention concerns an array comprising polynucleotides hybridizing to one or more of the following genes: STAT5A, STAT5B, WISP1, CKAP4, FGFR1, cdc25A, RASSF1, G-Catenin, H2AFZ, NME1, NRG1, BC12, TAGLN, YB1, Src, IGF1R, CD44, DIABLO, TIMP2, AREG, PDGFrA, CTSB, Hepsin, ErbB3, MTA, Gus, and VEGF, immobilized on a solid surface. The polynucleotides can be cDNA or oligonucleotides. The cDNAs are typically about 500 to 5000 bases long, while the oligonucleotides are typically about 20 to 80 bases long. An array can contain a very large number of cDNAs, or oligonucleotides, e.g. up to about 330,000 oligonucleotides. The solid surface presenting the array can, for example, be glass. The levels of the product of the gene transcripts can be measured by any technique known in the art, including, for example, immunohistochemistry or proteomics.

In various embodiments, the array comprises polynucleotides hybridizing to two at least two, at least three, at least four, at least five, at least six, at least seven, at least eight, at least nine, at least ten, at least eleven, at least twelve, at least thirteen, at least fourteen, at least fifteen, at least seventeen, at least eighteen, at least nineteen, at least twenty, at least twenty-one, at least twenty-two, at least twenty-three, at least twenty-four, at least twenty-five, at least twenty-six, or all twenty-seven of the genes listed above. In a particular embodiment, hybridization is performed under stringent conditions.

The invention further concerns a method of preparing a personalized genomics profile for a patient, comprising the steps of:

(a) subjecting RNA extracted from cancer tissue obtained from the patient to gene expression analysis;

(b) determining the expression level in the tissue of one or more genes selected from the group consisting of STAT5A, STAT5B, WISP1, CKAP4, FGFr1, cdc25A, RASSF1, G-Catenin, H2AFZ, NME1, NRG1, BC12, TAGLN, YB1, Src, IGF1R, CD44, DIABLO, TIMP2, AREG, PDGFRA, CTSB, Hepsin, ErbB3, MTA, Gus, and VEGF, wherein the expression level is normalized against a control gene or genes and optionally is compared to the amount found in a corresponding cancer reference tissue set; and

(c) creating a report summarizing the data obtained by said gene expression analysis.

The invention additionally concerns a method for amplification of a gene selected from the group consisting of STAT5A, STAT5B, WISP1, CKAP4, FGFr1, cdc25A, RASSF1, G-Catenin, H2AFZ, NME1, NRG1, BC12, TAGLN, YB1, Src, IGF1R, CD44, DIABLO, TIMP2, AREG, PDGFRA, CTSB, Hepsin, ErbB3, MTA, Gus, and VEGF by polymerase chain reaction (PCR), comprising performing said PCR by using a corresponding amplicon listed in Table 3, and a corresponding primer-probe set listed in Table 4.

The invention further encompasses any PCR primer-probe set listed in Tables 4, and any PCR amplicon listed in Table 3.

In yet another aspect, the invention concerns a prognostic method comprising:

(a) subjecting a sample comprising cancer cells obtained from a patient to quantitative analysis of the expression level of the RNA transcript of at least one gene selected from the group consisting of STAT5A, STAT5B, WISP1, CKAP4, FGFR1, cdc25A, RASSF1, G-Catenin, H2AFZ, NME1, NRG1, BC12, TAGLN, YB1, Src, IGF1R, CD44, DIABLO, TIMP2, AREG, PDGFRa, and CTSB, or their product, and

(b) identifying the patient as likely to have a decreased likelihood of responding well to treatment with an EGFR inhibitor if the normalized expression levels of said gene or genes, or their products, are elevated above a defined expression threshold.

In a further aspect, the invention concerns a prognostic method comprising:

(a) subjecting a sample comprising cancer cells obtained from a patient to quantitative analysis of the expression level of the RNA transcript of at least one gene selected from the group consisting of Hepsin, ErbB3, MTA, Gus, and VEGF or their product, and

(b) identifying the patient as likely to have an increased likelihood of responding well to treatment with an EGFR inhibitor if the normalized expression levels of said gene or genes, or their products, are elevated above a defined expression threshold.

### DETAILED DESCRIPTION OF THE PREFERRED EMBODIMENT

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a subject suffering from a disease, specifically cancer, such as breast cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and diseased subjects, or in various stages of disease development in a diseased subject.

The term “over-expression” with regard to an RNA transcript is used to refer the level of the transcript determined by normalization to the level of reference mRNAs, which might be all measured transcripts in the specimen or a particular reference set of mRNAs.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

The term “prognosis” is used herein to refer to the prediction of the likelihood of cancer-attributable death or progression, including recurrence, metastatic spread, and drug resistance, of a neoplastic disease, such as non-small cell lung cancer, or head and neck cancer. The term “prediction” is used herein to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

The term “long-term” survival is used herein to refer to survival for at least 1 year, more preferably for at least 2 years, most preferably for at least 5 years following surgery or other treatment.

The term “increased resistance” to a particular drug or treatment option, when used in accordance with the present invention, means decreased response to a standard dose of the drug or to a standard treatment protocol.

The term “decreased sensitivity” to a particular drug or treatment option, when used in accordance with the present invention, means decreased response to a standard dose of the drug or to a standard treatment protocol, where decreased response can be compensated for (at least partially) by increasing the dose of drug, or the intensity of treatment.

“Patient response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

The term “EGFR inhibitor” as used herein refers to a molecule having the ability to inhibit a biological function of a native epidermal growth factor receptor (EGFR). Accordingly, the term “inhibitor” is defined in the context of the biological role of EGFR. While preferred inhibitors herein specifically interact with (e.g. bind to) an EGFR, molecules that inhibit an EGFR biological activity by interacting with other members of the EGFR signal transduction pathway are also specifically included within this definition. A preferred EGFR biological activity inhibited by an EGFR inhibitor is associated with the development, growth, or spread of a tumor. EGFR inhibitors, without limitation, include peptides, non-peptide small molecules, antibodies, antibody fragments, antisense molecules, and oligonucleotide decoys.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like. In the context of the present invention, reference to “at least one,” “at least two,” “at least five,” etc. of the genes listed in any particular gene set means any one or any and all combinations of the genes listed.

The terms “expression threshold,” and “defined expression threshold” are used interchangeably and refer to the level of a gene or gene product in question above which the gene or gene product serves as a predictive marker for patient survival without cancer recurrence. The threshold is defined experimentally from clinical studies such as those described in the Example below. The expression threshold can be selected either for maximum sensitivity, or for maximum selectivity, or for minimum error. The determination of the expression threshold for any situation is well within the knowledge of those skilled in the art.

### B. Detailed Description

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

1. Gene Expression Profiling

In general, methods of gene expression profiling can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

2. Reverse Transcriptase PCR (RT-PCR)

Of the techniques listed above, the most sensitive and most flexible quantitative method is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, head and neck, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into CDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived CDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Cₜ).

To minimize errors and the effect of sample-to-sample variation, RT-PCR is usually performed using an internal standard. The ideal internal standard is expressed at a relatively constant level among different tissues, and is unaffected by the experimental treatment. RNAs frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles {for example: T. E. Godfrey et al. J. Molec. Diagnostics 2; 84-91 [2000]; K. Specht et al., Am. J. Pathol. 158: 419-29 [2001]}. Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR.

3. Microarrays

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled CDNA probes may be generated through incorporation of fluorescent nucleotides by reverse transcription of RNA extracted from tissues of interest. Labeled CDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. NatL Acad. Sci. USA 93(2):106-149 (1996)). Microarray analysis can be performed by commercially available equipment, following manufacturer's protocols, such as by using the Affymetrix GenChip technology, or Agilent's microarray technology.

The development of microarray methods for large-scale analysis of gene expression makes it possible to search systematically for molecular markers of cancer classification and outcome prediction in a variety of tumor types.

4. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al, Cell 88:243-51 (1997).

5. Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

6. Immunohistochemistry

Immunohistochemistry methods are also suitable for detecting the expression levels of the prognostic markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

7. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

8. EGFR Inhibitors

The epidermal growth factor receptor (EGFR) family (which includes EGFR, erb-B2, erb-B3, and erb-B4) is a family of growth factor receptors that are frequently activated in epithelial malignancies. Thus, the epidermal growth factor receptor (EGFR) is known to be active in several tumor types, including, for example, ovarian cancer, pancreatic cancer, non-small cell lung cancer {NSCLC}, breast cancer, and head and neck cancer. Several EGFR inhibitors, such as ZD1839 (also known as gefitinib or Iressa); and OS1774 (Erlotinib, Tarceva™), are promising drug candidates for the treatment of cancer.

Iressa, a small synthetic quinazoline, competitively inhibits the ATP binding site of EGFR, a growth-promoting receptor tyrosine kinase, and has been in Phase III clinical trials for the treatment of non-small-cell lung carcinoma. Another EGFR inhibitor, [agr]cyano-[bgr]methyl-N-[(trifluoromethoxy)phenyl]-propenamide (LFM-A12), has been shown to inhibit the proliferation and invasiveness of human breast cancer cells.

Cetuximab is a monoclonal antibody that blocks the EGFR and EGFR-dependent cell growth. It is currently being tested in phase III clinical trials.

Tarceva™ has shown promising indications of anti-cancer activity in patients with advanced ovarian cancer, and non-small cell lung and head and neck carcinomas.

The present invention provides valuable molecular markers that predict whether a patient who is a candidate for treatment with an EGFR inhibitor drug is likely to respond to treatment with an EGFR inhibitor.

The listed examples of EGFR inhibitors represent both small organic molecule and anti-EGFR antibody classes of drugs. The findings of the present invention are equally applicable to other EGFR inhibitors, including, without limitation, antisense molecules, small peptides, etc.

9. General Description of the mRNA Isolation, Purification and Amplification

The steps of a representative protocol for profiling gene expression using fixed, paraffin-embedded tissues as the RNA source, including mRNA isolation, purification, primer extension and amplification are given in various published journal articles {for example: T. E. Godfrey et al. J. Molec. Diagnostics 2: 84-91 [2000]; K. Specht et al., Am. J. Pathol. 158: 419-29 [2001]}. Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR. Finally, the data are analyzed to identify the best treatment option(s) available to the patient on the basis of the characteristic gene expression pattern identified in the tumor sample examined.

10. Cancer Gene Set, Assayed Gene Subsequences, and Clinical Application of Gene Expression Data

An important aspect of the present invention is to use the measured expression of certain genes by cancer (e.g. lung cancer) tissue to provide prognostic information. For this purpose it is necessary to correct for (normalize away) both differences in the amount of RNA assayed and variability in the quality of the RNA used. Therefore, the assay typically measures and incorporates the expression of certain normalizing genes, including well known housekeeping genes, such as GAPDH and Cyp1. Alternatively, normalization can be based on the mean or median signal (Ct) of all of the assayed genes or a large subset thereof (global normalization approach). On a gene-by-gene basis, measured normalized amount of a patient tumor mRNA is compared to the amount found in a cancer tissue reference set. The number (N) of cancer tissues in this reference set should be sufficiently high to ensure that different reference sets (as a whole) behave essentially the same way. If this condition is met, the identity of the individual cancer tissues present in a particular set will have no significant impact on the relative amounts of the genes assayed. Usually, the cancer tissue reference set consists of at least about 30, preferably at least about 40 different FPE cancer tissue specimens. Unless noted otherwise, normalized expression levels for each mRNA/tested tumor/patient will be expressed as a percentage of the expression level measured in the reference set. More specifically, the reference set of a sufficiently high number (e.g. 40) of tumors yields a distribution of normalized levels of each mRNA species. The level measured in a particular tumor sample to be analyzed falls at some percentile within this range, which can be determined by methods well known in the art. Below, unless noted otherwise, reference to expression levels of a gene assume normalized expression relative to the reference set although this is not always explicitly stated.

Further details of the invention will be apparent from the following non-limiting Example.

### Example

### A Phase II Study of Gene Expression in Non-Small Cell Lung Cancer (NSCL)

A gene expression study was designed and conducted with the primary goal to molecularly characterize gene expression in paraffin-embedded, fixed tissue samples of NSCLC patients who did or did not respond to treatment with an EGFR inhibitor. The results are based on the use of one EGFR inhibitor.

### Study Design

Molecular assays were performed on paraffin-embedded, formalin-fixed tumor tissues obtained from 29 individual patients diagnosed with NSCLC. Patients were included in the study only if histopathologic assessment, performed as described in the Materials and Methods section, indicated adequate amounts of tumor tissue. All patients had a history of prior treatment for NSCLC, and the nature of pretreatment varied.

### Materials and Methods

Each representative tumor block was characterized by standard histopathology for diagnosis, semi-quantitative assessment of amount of tumor, and tumor grade. A total of 6 sections (10 microns in thickness each) were prepared and placed in two Costar Brand Microcentrifuge Tubes (Polypropylene, 1.7 mL tubes, clear; 3 sections in each tube). If the tumor constituted less than 30% of the total specimen area, the sample may have been dissected by the pathologist, putting the tumor tissue directly into the Costar tube.

If more than one tumor block was obtained as part of the surgical procedure, the block most representative of the pathology was used for analysis.

### Gene Expression Analysis

mRNA was extracted and purified from fixed, paraffin-embedded tissue samples, and prepared for gene expression analysis as described above.

Molecular assays of quantitative gene expression were performed by RT-PCR, using the ABI PRISM 7900™ Sequence Detection System™ (Perkin-Elmer-Applied Biosystems, Foster City, Calif., USA). ABI PRISM 7900™ consists of a thermocycler, laser, charge-coupled device (CCD), camera and computer. The system amplifies samples in a 384-well format on a thermocycler. During amplification, laser-induced fluorescent signal is collected in real-time through fiber optics cables for all 384 wells, and detected at the CCD. The system includes software for running the instrument and for analyzing the data.

### Analysis and Results

Tumor tissue was analyzed for 185 cancer-related genes and 7 reference genes. The threshold cycle (CT) values for each patient were normalized based on the mean of all genes for that particular patient. Clinical outcome data were available for all patients.

Outcomes were evaluated in two ways, each breaking patients into two groups with respect to response.

One analysis categorized complete or partial response [RES] as one group, and stable disease (min of 3 months) or progressive disease as the other group [NR]. The second analysis grouped patients with respect to clinical benefit, where clinical benefit was defined as partial response, complete response, or stable disease at 3 months.

Response (partial response and complete response) was determined by the Response Evaluation Criteria In Solid Tumors (RECIST criteria). Stable disease was designated as the absence of aggressive disease for 3 or more months.

Analysis of 17 Patients by T-Test

Analysis was performed on all 17 treated patients to determine the relationship between normalized gene expression and the binary outcomes of RES (response) or NR (non-response). At test was performed on the group of patients classified as RES or NR and the p-values for the differences between the groups for each gene were calculated. The following table lists the 23 genes for which the p-value for the differences between the groups was <0.10. In this case response was defined as a partial or complete response, the former being >50% shrink of the tumor and the latter being disappearance of the tumor. As shown, response was identified in two patients.

TABLE 1

| | No Resp | Yes Resp | | | | No Resp | Yes Resp |
|---|---|---|---|---|---|---|---|
| | Mean | Mean | t-value | df | p | Valid N | Valid N |
| STAT5A.1 | −0.9096 | −2.1940 | 3.48829 | 15 | 0.003302 | 15 | 2 |
| STAT5B.2 | −0.9837 | −2.2811 | 3.35057 | 15 | 0.004380 | 15 | 2 |
| WISP1.1 | −3.8768 | −6.1318 | 2.88841 | 15 | 0.011256 | 15 | 2 |
| CKAP4.2 | −0.1082 | −1.0934 | 2.54034 | 15 | 0.022627 | 15 | 2 |
| FGFR1.3 | −3.0647 | −4.9591 | 2.42640 | 15 | 0.028323 | 15 | 2 |
| cdc25A.4 | −4.3752 | −5.2888 | 2.28383 | 15 | 0.037373 | 15 | 2 |
| RASSF1.3 | −1.8402 | −2.8002 | 2.28308 | 15 | 0.037427 | 15 | 2 |
| ErbB3.1 | −10.0166 | −8.7599 | −2.13036 | 15 | 0.050103 | 15 | 2 |
| GUS.1 | −2.2284 | −1.2524 | −2.12833 | 15 | 0.050296 | 15 | 2 |
| NRG1.3 | −7.6976 | −10.2172 | 2.10836 | 15 | 0.052227 | 15 | 2 |
| Bcl2.2 | −2.4212 | −3.9768 | 2.10197 | 15 | 0.052859 | 15 | 2 |
| Hepsin.1 | −7.2602 | −5.0055 | −2.09847 | 15 | 0.053208 | 15 | 2 |
| CTSB.1 | 3.2027 | 2.0683 | 2.06857 | 15 | 0.056279 | 15 | 2 |
| TAGLN.3 | 1.7465 | 0.0009 | 2.05991 | 15 | 0.057199 | 15 | 2 |
| YB-1.2 | 1.3480 | 0.8782 | 2.03095 | 15 | 0.060374 | 15 | 2 |
| Src.2 | −0.0393 | −0.9239 | 1.93370 | 15 | 0.072248 | 15 | 2 |
| IGF1R.3 | −2.8269 | −3.7970 | 1.93140 | 15 | 0.072553 | 15 | 2 |
| CD44s.1 | 0.0729 | −1.3075 | 1.90370 | 15 | 0.076315 | 15 | 2 |
| DIABLO.1 | −3.6865 | −4.4254 | 1.84770 | 15 | 0.084461 | 15 | 2 |
| VEGF.1 | 1.3981 | 2.3817 | −1.82941 | 15 | 0.087285 | 15 | 2 |
| TIMP2.1 | 2.5347 | 1.4616 | 1.82763 | 15 | 0.087565 | 15 | 2 |
| AREG.2 | −1.5665 | −4.5616 | 1.82558 | 15 | 0.087887 | 15 | 2 |
| PDGFRa.2 | −0.8243 | −2.7529 | 1.79553 | 15 | 0.092738 | 15 | 2 |

In the foregoing Table 1, lower mean expression Cₜ values indicate lower expression and, conversely, higher mean expression values indicate higher expression of a particular gene. Thus, for example, expression of the STAT5A or STAT5B gene was higher in patients who did not respond to EGFR inhibitor treatment than in patients that did respond to the treatment. Accordingly, elevated expression of STAT5A or STAT5B is an indication of poor outcome of treatment with an EGFR inhibitor. Phrasing it differently, if the STAT5A or STAT5B gene is over-expressed in a tissue simple obtained from the cancer of a NSCLC patient, treatment with an EGFR inhibitor is not likely to work, therefore, the physician is well advised to look for alternative treatment options.

Accordingly, the elevated expression of STAT5A, STAT5B, WISP1, CKAP4, FGFR1, cdc25A or RASSF1 in a tumor is an indication that the patient is not likely to respond well to treatment with an EGFR inhibitor. On the other hand, elevated expression of ErbB3 is an indication that the patient is likely to respond to EGFR inhibitor treatment.

In Table 2 below the binary analysis was carried with respect to clinical benefit, defined as either partial response, complete response, or stable disease. As shown, 5 patients met these criteria for clinical benefit.

TABLE 2

| | No | Yes | | | | No | Yes |
|---|---|---|---|---|---|---|---|
| | Benefit | Benefit | | | | Benefit | Benefit |
| | Mean | Mean | t-value | df | p | Valid N | Valid N |
| G-Catenin.1 | 0.0595 | −0.7060 | 2.28674 | 15 | 0.037164 | 12 | 5 |
| Hepsin.1 | −7.4952 | −5.7945 | −2.28516 | 15 | 0.037277 | 12 | 5 |
| ErbB3.1 | −10.1269 | −9.2493 | −2.09612 | 15 | 0.053444 | 12 | 5 |
| MTA1.1 | −2.3587 | −1.6977 | −1.94548 | 15 | 0.070705 | 12 | 5 |
| H2AFZ.2 | −1.0432 | −1.6448 | 1.82569 | 15 | 0.087869 | 12 | 5 |
| NME1.3 | 0.4774 | −0.1769 | 1.80874 | 15 | 0.090578 | 12 | 5 |
| LMYC.2 | −3.6259 | −3.2175 | −1.71006 | 15 | 0.107853 | 12 | 5 |
| AREG.2 | −1.3375 | −3.3140 | 1.67977 | 15 | 0.113704 | 12 | 5 |
| Surfact A1.1 | −1.9341 | 2.9822 | −1.63410 | 15 | 0.123046 | 12 | 5 |
| CDH1.3 | −1.3614 | −2.1543 | 1.59764 | 15 | 0.130971 | 12 | 5 |
| PTPD1.2 | −2.7517 | −2.0708 | −1.52929 | 15 | 0.147004 | 12 | 5 |

As shown in the above Table 2, 6 genes correlated with clinical benefit with p<0.1 Expression of G-catenin, H2AFZ, and NME1 was higher in patients who did not respond to anti-EGFR treatment. Thus, greater expression of these genes is an indication that patients are unlikely to benefit from anti-EGFR treatment. Conversely, expression of Hepsin, ErbB3, and MTA was higher in patients who did respond to anti-EGFR treatment. Greater expression of these genes indicates that patients are likely to benefit from anti-EGFR treatment.

Table 3 shows the accession numbers and amplicon sequences used during the PCR amplification of the genes identified.

Table 4 shows the accession numbers and the sequences of the primer/probe sets used during the PCR amplification of the genes identified. For each gene the forward primer sequence is identified as f2, the probe sequence as p2, and the reverse primer sequence as r2.

It is emphasized that while the data presented herein were obtained using tissue samples from NSCLC, the conclusions drawn from the tissue expression profiles are equally applicable to other cancers, such as, for example, colon cancer, ovarian cancer, pancreatic cancer, breast cancer, and head and neck cancer.

All references cited throughout the specification are hereby expressly incorporated by reference.

TABLE 3

| | | | | | |
|---|---|---|---|---|---|
| SEQ | | | Gene | Gene | |
| ID | Gene | Accession | Sequence | Sequence | |
| NO: | Name | Number | Start | Stop | Sequence |

| | | | | | |
|---|---|---|---|---|---|
| 1 | AREG | NM_001657 | 404 | 486 | TGTGAGTGAA |
| | | | | | ATGCCTTCTA |
| | | | | | GTAGTGAACC |
| | | | | | GTCCTCGGGA |
| | | | | | GCCGACTATG |
| | | | | | ACTACTCAGA |
| | | | | | AGAGTATGAT |
| | | | | | AACGAACCA |
| | | | | | CAA |
| 2 | Bcl2 | NM_000633 | 1386 | 1459 | CAGATGGACC |
| | | | | | TAGTACCCAC |
| | | | | | TGAGATTTCC |
| | | | | | ACGCCGAAGG |
| | | | | | ACAGCGATGG |
| | | | | | GAAAAATGCC |
| | | | | | CTTAAATCAT |
| | | | | | AGG |
| 3 | CD44s | M59040 | 644 | 722 | GACGAAGACA |
| | | | | | GTCCCTGGAT |
| | | | | | CACCGACAGC |
| | | | | | ACAGACAGAA |
| | | | | | TCCCTGCTAC |
| | | | | | CAGAGACCAA |
| | | | | | GACACATTCC |
| | | | | | ACCCCAGT |
| 4 | cdc25 | NM_001789 | 2203 | 2274 | TCTTGCTGGC |
| | | | | | TACGCCTCTT |
| | | | | | CTGTCCCTGT |
| | | | | | TAGACGTCCT |
| | | | | | CCGTCCATAT |
| | | | | | CAGAACTGTG |
| | | | | | CCACAATG |
| | | | | | CAG |
| 5 | CKAP4 | NM_006825 | 1702 | 1768 | AAAGCCTCAG |
| | | | | | TCAGCCAAGT |
| | | | | | GGAGGCGGAC |
| | | | | | TTGAAAATGC |
| | | | | | TCAGGACTGC |
| | | | | | TGTGGACAGT |
| | | | | | TTGGTT |
| 6 | CTSB | NM_001908 | 897 | 959 | GGCCGAGATC |
| | | | | | TACAAAAACG |
| | | | | | GCCCCGTGGA |
| | | | | | GGGAGCTTTC |
| | | | | | TCTGTGTATT |
| | | | | | CGGACTTCCT |
| | | | | | GC |
| 7 | DIABLO | NM_019887 | 16 | 89 | CACAATGGCG |
| | | | | | GCTCTGAAGA |
| | | | | | GTTGGCTGTC |
| | | | | | GCGCAGCGTA |
| | | | | | ACTTCATTCT |
| | | | | | TCAGGTACAG |
| | | | | | ACAGTGTTTG |
| | | | | | TGT |
| 8 | ErbB3 | NM_001982 | 3669 | 3750 | CGGTTATGTC |
| | | | | | ATGCCAGATA |
| | | | | | CACACCTCAA |
| | | | | | AGGTACTCCC |
| | | | | | TCCTCCCGGG |
| | | | | | AAGGCACCCT |
| | | | | | TTCTTCAGTG |
| | | | | | GGTCTCAGT |
| | | | | | CTC |
| 9 | FDFR1 | NM_023109 | 2685 | 2759 | CACGGGACAT |
| | | | | | TCACCACATC |
| | | | | | GACTACTATA |
| | | | | | AAAAGACAAC |
| | | | | | CAACGGCCGA |
| | | | | | CTGCCTGTGA |
| | | | | | AGTGGATGGC |
| | | | | | ACCC |
| 10 | G-Catenin | NM_002230 | 229 | 297 | TCAGCAGCAA |
| | | | | | GGGCATCATG |
| | | | | | GAGGAGGATG |
| | | | | | AGGCCTGCGG |
| | | | | | GCGCCAGTAC |
| | | | | | ACGCTCAAGA |
| | | | | | AAACCACC |
| 11 | GUS | NM_000181 | 1933 | 2006 | CCCACTCAGT |
| | | | | | AGCCAAGTCA |
| | | | | | CAATGTTTGG |
| | | | | | AAAACAGCCC |
| | | | | | GTTTACTTGA |
| | | | | | GCAAGACTGA |
| | | | | | TACCACCTGC |
| | | | | | GTG |
| 12 | H2AFZ | NM_002106 | 135 | 206 | CCGGAAAGGC |
| | | | | | CAAGACAAAG |
| | | | | | GCGGTTTCCC |
| | | | | | GCTCGCAGAG |
| | | | | | AGCCGGCTTG |
| | | | | | CAGTTCCCAG |
| | | | | | TGGGCCGT |
| | | | | | ATT |
| 13 | Hepsin | NM_002151 | 633 | 717 | AGGCTGCTGG |
| | | | | | AGGTCATCTC |
| | | | | | CGTGTGTGAT |
| | | | | | TGCCCCAGAG |
| | | | | | GCCGTTTCTT |
| | | | | | GGCCGCCATC |
| | | | | | TGCCAAGACT |
| | | | | | GTGGCCGCAG |
| | | | | | GAAG |
| 14 | IGF1R | NM_000875 | 3467 | 3550 | GCATGGTAGC |
| | | | | | CGAAGATTTC |
| | | | | | ACAGTCAAAA |
| | | | | | TCGGAGATTT |
| | | | | | TGGTATGACG |
| | | | | | CGAGATATCT |
| | | | | | ATGAGACAGA |
| | | | | | CTATTACCGG |
| | | | | | AAA |
| 15 | MTA1 | NM_004689 | 2258 | 2335 | CCGCCCTCAC |
| | | | | | CTGAAGAGAA |
| | | | | | ACGCGCTCCT |
| | | | | | TGGCGGACAC |
| | | | | | TGGGGGAGGA |
| | | | | | GAGGAAGAAG |
| | | | | | CGCGGCTAAC |
| | | | | | TTATTCC |
| 16 | NME1 | NM_000269 | 365 | 439 | CCAACCCTGC |
| | | | | | AGACTCCAAG |
| | | | | | CCTGGGACCA |
| | | | | | TCCGTGGAGA |
| | | | | | CTTCTGCATA |
| | | | | | CAAGTTGGCA |
| | | | | | GGAACATTAT |
| | | | | | ACAT |
| 17 | NRG1 | NM_013957 | 1697 | 1780 | CGAGACTCTC |
| | | | | | CTCATAGTGA |
| | | | | | AAGGTATGTG |
| | | | | | TCAGCCATGA |
| | | | | | CCACCCCGGC |
| | | | | | TCGTATGTCA |
| | | | | | CCTGTAGATT |
| | | | | | TCCACACGCC |
| | | | | | AAG |
| 18 | PDGFRa | NM_006206 | 2151 | 2223 | GGGAGTTTCC |
| | | | | | AAGAGATGGA |
| | | | | | CTAGTGCTTG |
| | | | | | GTCGGGTCTT |
| | | | | | GGGGTCTGGA |
| | | | | | GCGTTTGGGA |
| | | | | | AGGTGGTTGA |
| | | | | | AG |
| 19 | RASSF1 | NM_007182 | 409 | 478 | AGTGGGAGAC |
| | | | | | ACCTGACCTT |
| | | | | | TCTCAAGCTG |
| | | | | | AGATTGAGCA |
| | | | | | GAAGATCAAG |
| | | | | | GAGTACAATG |
| | | | | | CCCAGATCA |
| 20 | Src | NM_004383 | 979 | 1043 | CCTGAACATG |
| | | | | | AAGGAGCTGA |
| | | | | | AGCTGCTGCA |
| | | | | | GACCATCGGG |
| | | | | | AAGGGGGAGT |
| | | | | | TCGGAGACGT |
| | | | | | GATG |
| 21 | STAT5A | NM_003152 | 2165 | 2242 | GAGGCGCTCA |
| | | | | | ACATGAAATT |
| | | | | | CAAGGCCGAA |
| | | | | | GTGCAGAGCA |
| | | | | | ACCGGGGCCT |
| | | | | | GACCAAGGAG |
| | | | | | AACCTCGTGT |
| | | | | | TCCTGGC |
| 22 | STAT5B | NM_012448 | 1539 | 1613 | CCAGTGGTGG |
| | | | | | TGATCGTTCA |
| | | | | | TGGCAGCCAG |
| | | | | | GACAACAATG |
| | | | | | CGACGGCCAC |
| | | | | | TGTTCTCTGG |
| | | | | | GACAATGCTT |
| | | | | | TTGC |
| 23 | TAGLN | NM_003186 | 345 | 418 | GATGGAGCAG |
| | | | | | GTGGCTCAGT |
| | | | | | TCCTGAAGGC |
| | | | | | GGCTGAGGAC |
| | | | | | TCTGGGGTCA |
| | | | | | TCAAGACTGA |
| | | | | | CATGTTCCAG |
| | | | | | ACT |
| 24 | TIMP2 | NM_003255 | 673 | 742 | TCACCCTCTG |
| | | | | | TGACTTCATC |
| | | | | | GTGCCCTGGG |
| | | | | | ACACCCTGAG |
| | | | | | CACCACCCAG |
| | | | | | AAGAAGAGCC |
| | | | | | TGAACCACA |
| 25 | VFGF | NM_003376 | 26 | 97 | CTGCTGTCTT |
| | | | | | GGGTGCATTG |
| | | | | | GAGCCTTGCC |
| | | | | | TTGCTGCTCT |
| | | | | | ACCTCCACCA |
| | | | | | TGCCAAGTGG |
| | | | | | TCCCAGGC |
| | | | | | TGC |
| 26 | W1SP1 | NM_003882 | 913 | 988 | AGAGGCATCC |
| | | | | | ATGAACTTCA |
| | | | | | CACTTGCGGG |
| | | | | | CTGCATCAGC |
| | | | | | ACACGCTCCT |
| | | | | | ATCAACCCAA |
| | | | | | GTACTGTGGA |
| | | | | | GTTTG |
| 27 | YB-1 | NM_004559 | 551 | 627 | AGACTGTGGA |
| | | | | | GTTTGATGTT |
| | | | | | GTTGAAGGAG |
| | | | | | AAAAGGGTGC |
| | | | | | GGAGGCAGCA |
| | | | | | AATGTTACAG |
| | | | | | GTCCTGGTGG |
| | | | | | TGTTCC |

TABLE 4

| | | | | | |
|---|---|---|---|---|---|
| SEQ ID | Gene | Accession | | | |
| NO: | Name | Number | Part Name | Sequence | Length |

| | | | | | |
|---|---|---|---|---|---|
| 28 | AREG | NM-001657 | S0025/AREG.f2 | TGTGAGTGAAATGCCTTCTAGTAGTGA | 27 |
| 29 | AREG | NM-001657 | S0026/AREG.p2 | CCGTCCTCGGGAGCCGACTATGA | 23 |
| 30 | AREG | NM-001657 | S0027/AREG.r2 | TTGTGGTTCGTTATCATACTCTTCTGA | 27 |
| 31 | Bc12 | NM-000633 | S0043IBc12.f2 | CAGATGGACCTAGTACCCACTGAGA | 25 |
| 32 | Bc12 | NM-000633 | S0044/Bc12.p2 | TTCCACGCCGAAGGACAGCGAT | 22 |
| 33 | Bc12 | NM-000633 | S0045fBc12.r2 | CCTATGATTTAAGGGCATTTTTCC | 24 |
| 34 | CD44s | M59040 | S3102/CD44s.f1 | GACGAAGACAGTCCCTGGAT | 20 |
| 35 | CD44s | M59040 | S3103/CD44s.r1 | ACTGGGGTGGAATGTGTCTT | 20 |
| 36 | CD44s | M59040 | S3104/CD44s.p1 | CACCGACAGCACAGACAGAATCCC | 24 |
| 37 | cdc25A | NM-001789 | S0070/cdc25A.f4 | TCTTGCTGGCTACGCCTCTT | 20 |
| 38 | cdc25A | NM-001789 | S0071/cdc25A.p4 | TGTCCCTGTTAGACGTCCTCCGTCCATA | 28 |
| 39 | cdc25A | NM-001789 | S0072/cdc25A.r4 | CTGCATTGTGGCACAGTTCTG | 21 |
| 40 | CKAP4 | NM-006825 | S2381/CKAP4.f2 | AAAGCCTCAGTCAGCCAAGT | 20 |
| 41 | CKAP4 | NM-006825 | S2382/CKAP4.r2 | AACCAAACTGTCCACAGCAG | 20 |
| 42 | CKAP4 | NM-006825 | S2383/CKAP4.p2 | TCCTGAGCATTTTCAAGTCCGCCT | 24 |
| 43 | CTSB | NM-001908 | S1146/CTSB.f1 | GGCCGAGATCTACAAAAACG | 20 |
| 44 | CTSB | NM-001908 | S1147/CTSB.r1 | GCAGGAAGTCCGAATACACA | 20 |
| 45 | CTSB | NM-001908 | S1180/CTSB.p1 | CCCCGTGGAGGGAGCTTTCTC | 21 |
| 46 | DIABLO | NM-019887 | S0808/DIABLO.f1 | CACAATGGCGGCTCTGAAG | 19 |
| 47 | DIABLO | NM-019887 | S0809/DIABLO.r1 | ACACAAACACTGTCTGTACCTGAAGA | 26 |
| 48 | DIABLO | NM-019887 | S1105/DIABLO.p1 | AAGTTACGCTGCGCGACAGCCAA | 23 |
| 49 | ErbB3 | NM-001982 | S0112/ErbB3.f1 | CGGTTATGTCATGCCAGATACAC | 23 |
| 50 | ErbB3 | NM-001982 | S0113/ErbB3.p1 | CCTCAAAGGTACTCCCTCCTCCCGG | 25 |
| 51 | ErbB3 | NM-001982 | S0114/ErbB3.r1 | GAACTGAGACCCACTGAAGAAAGG | 24 |
| 52 | FGFR1 | NM-023109 | S0818/FGFR1.f3 | CACGGGACATTCACCACATC | 20 |
| 53 | FGFR1 | NM-023109 | S0819/FGFR1.r3 | GGGTGCCATCCACTTCACA | 19 |
| 54 | FGFR1 | NM-023109 | S1110/FGFR1.p3 | ATAAAAAGACAACCAACGGCCGACTGC | 27 |
| 55 | G-Catenin | NM-00223O | S2153/G-Cate.f1 | TCAGCAGCAAGGGCATCAT | 19 |
| 56 | G-Catenin | NM-002230 | S2154/G-Cate.r1 | GGTGGTTTTCTTGAGCGTGTACT | 23 |
| 57 | G-Catenin | NM-002230 | S2155/G-Cate.p1 | CGCCCGCAGGCCTCATCCT | 19 |
| 58 | GUS | NM-000181 | S0139/GUS.f1 | CCCACTCAGTAGCCAAGTCA | 20 |
| 59 | GUS | NM-000181 | S0140/GUS.p1 | TCAAGTAAACGGGCTGTTTTCCAAACA | 27 |
| 60 | GUS | NM-000181 | S0141/GUS.r1 | CACGCAGGTGGTATCAGTCT | 20 |
| 61 | H2AFZ | NM-002106 | S3012/H2AFZ.f2 | CCGGAAAGGCCAAGACAA | 18 |
| 62 | H2AFZ | NM-002106 | S3013/H2AFZ.r2 | AATACGGCCCACTGGGAACT | 20 |
| 63 | H2AFZ | NM-002106 | S3014/H2AFZ.p2 | CCCGCTCGCAGAGAGCCGG | 19 |
| 64 | Hepsin | NM-002151 | S2269/Hepsin.f1 | AGGCTGCTGGAGGTCATCTC | 20 |
| 65 | Hepsin | NM-002151 | S2270/Hepsin.r1 | CTTCCTGCGGCCACAGTCT | 19 |
| 66 | Hepsin | NM-002151 | S2271/Hepsin.p1 | CCAGAGGCCGTTTCTTGGCCG | 21 |
| 67 | IGF1R | NM-000875 | S1249/IGF1Rf3 | GCATGGTAGCCGAAGATTTCA | 21 |
| 68 | IGF1R | NM-000875 | S1250/IGF1R.r3 | TTTCCGGTAATAGTCTGTCTCATAGATATC | 30 |
| 69 | IGF1R | NM-000875 | S1251/IGF1R.p3 | CGCGTCATACCAAAATCTCCGATTTTGA | 28 |
| 70 | MTA1 | NM-004689 | S2369/MTA1.f1 | CCGCCCTCACCTGAAGAGA | 19 |
| 71 | MTA1 | NM-004689 | S2370/MTA1.r1 | GGAATAAGTTAGCCGCGCTTCT | 22 |
| 72 | MTA1 | NM-004689 | S2371/MTA1.p1 | CCCAGTGTCCGCCAAGGAGCG | 21 |
| 73 | NME1 | NM-000269 | S2526/NME1.f3 | CCAACCCTGCAGACTCCAA | 19 |
| 74 | NMEI | NM-000269 | S2527/NME1.r3 | ATGTATAATGTTCCTGCCAACTTGTATG | 28 |
| 75 | NMEI | NM-000269 | S2528/NME1.p3 | CCTGGGACCATCCGTGGAGACTTCT | 25 |
| 76 | NRG1 | NM-013957 | S1240/NRG1.f3 | CGAGACTCTCCTCATAGTGAAAGGTAT | 27 |
| 77 | NRG1 | NM-013957 | S1241/NRG1.r3 | CTTGGCGTGTGGAAATCTACAG | 22 |
| 78 | NRG1 | NM-013957 | S1242/NRG1.p3 | ATGACCACCCCGGCTCGTATGTCA | 24 |
| 79 | PDGFRa | NM-006206 | S0226/PDGFRa.f2 | GGGAGTTTCCAAGAGATGGA | 20 |
| 80 | PDGFRa | NM-006206 | S0227/PDGFRa.p2 | CCCAAGACCCGACCAAGCACTAG | 23 |
| 81 | PDGFRa | NM-006206 | S0228/PDGFRa.r2 | CTTCAACCACCTTCCCAIAAC | 20 |
| 82 | RASSF1 | NM-007182 | S2393/RASSF1.f3 | AGTGGGAGACACCTGACCTT | 20 |
| 83 | RASSF1 | NM-007182 | S2394/RASSF1.r3 | TGATCTGGGCATTGTACTCC | 20 |
| 84 | RASSE1 | NM-007182 | S2395/RASSF1.p3 | TTGATCTTCTGCTCAATCTCAGCTTGAGA | 29 |
| 85 | Src | NM-004383 | S1820/Src.f2 | CCTGAACATGAAGGAGCTGA | 20 |
| 86 | Src | NM-004383 | S1821/Src.r2 | CATCACGTCTCCGAACTCC | 19 |
| 87 | Src | NM-004383 | S1822/Src.p2 | TCCCGATGGTCTGCAGCAGCT | 21 |
| 88 | STAT5A | NM-003152 | S1219/STAT5A.f1 | GAGGCGCTCAACATGAAATTC | 21 |
| 89 | STAT5A | NM-003152 | S1220/STAT5A.r1 | GCCAGGAACACGAGGTTCTC | 20 |
| 90 | STAT5A | NM-003152 | S1221/STAT5A.p1 | CGGTTGCTCTGCACTTCGGCCT | 22 |
| 91 | STAT5B | NM-012448 | S2399/STAT5B.f2 | CCAGTGGTGGTGATcGTTcA | 20 |
| 92 | STAT5B | NM-012448 | S2400/STAT5B.r2 | GCAAAAGCATTGTCCCAGAGA | 21 |
| 93 | STAT5B | NM-012448 | S2401/STAT5B.p2 | CAGCCAGGACAACAATGCGACGG | 23 |
| 94 | TAGLN | NM-003186 | S3185/TAGLN.f3 | GATGGAGCAGGTGGCTCAGT | 20 |
| 95 | TAGLN | NM-003186 | S3186/TAGLN.r3 | AGTCTGGAACATGTCAGTCTTGATG | 25 |
| 96 | TAGLN | NM-003186 | 83187/TAGLN.p3 | CCCAGAGTCCTCAGCCGCCTTCAG | 24 |
| 97 | TIMP2 | NM-003255 | S1680/TIMP2.f1 | TCACCCTCTGTGACTTCATCGT | 22 |
| 98 | TIMP2 | NM-003255 | S1681/TIMP2.r1 | TGTGGTTCAGGCTCTTCTTCTG | 22 |
| 99 | TIMP2 | NM-003255 | S1682/TIMP2.p1 | CCCTGGGACACCCTGAGCACCA | 22 |
| 100 | VEGF | NM-003376 | S0286VEGF.f1 | CTGCTGTCTTGGGTGCATTG | 20 |
| 101 | VEGF | NM-003376 | S0287VEGF.p1 | TTGCCTTGCTGCTCTACCTCCACCA | 25 |
| 102 | VEGF | NM-003376 | S0288VEGF.r1 | GCAGCCTGGGACCACTTG | 18 |
| 103 | W1SP1 | NM-003882 | S1671/WISP1.f1 | AGAGGCATCCATGAACTTCACA | 22 |
| 104 | W1SP1 | NM-003882 | S1672/WISP1.r1 | CAAACTCCACAGTACTTGGGTTGA | 24 |
| 105 | W1SP1 | NM-003882 | S16731WISP1.p1 | CGGGCTGCATCAGCACACGC | 20 |
| 106 | YB-1 | NM-004559 | S1194/YB-1.f2 | AGACTGTGGAGTTTGATGTTGTTGA | 25 |
| 107 | YB-1 | NM-004559 | S1195NB-1.r2 | GGAACACCACCAGGACCTGTAA | 22 |
| 108 | YB-1 | NM-004559 | S1199/YB-1.p2 | TTGCTGCCTCCGCACCCTTTTCT | 23 |

What is claimed is:

1. A method for predicting the likelihood that a patient who is a candidate for treatment with an EGFR inhibitor will respond to said treatment, comprising determining the expression level of the transcript of STAT5B, or its expression product, in cancer cells obtained from said patient, wherein over-expression of the transcript of STAT5B normalized relative to the mean level of the RNA transcripts, or the corresponding expression products, in a cancer tissue reference set of at least 30 cancer tissue specimens or the product of two or more reference normalizing genes, indicates that said treatment-is not likely to provide a benefit to the patient.

2. The method of claim 1 further comprising determining the expression level of one or more prognostic RNA transcripts or their expression products wherein the prognostic transcript is the transcript of one or more genes selected from the group consisting of: STAT5A, WISP1, CKAP4, FGFR1, cdc25A, RASSF1, G-Catenin, H2AFZ, NME1, NRG1, BC12, TAGLN, YB-1, Src, IGF1R, CD44, DIABLO, TIMP2, PDGFRa, CTSB, Hepsin, ErbB3, MTA1, Gus, and VEGF, wherein (a) over-expression of the transcript, or the corresponding expression product, of one or more of STAT5A, STAT5B, WISP1, CKAP4, FGFR1, cdc25A, RASSF1, G-Catenin, H2AFZ, NME1, NRG1, BC12, TAGLN, YB1, Src, IGF1R, CD44, DIABLO, TIMP2, PDGFRa, and CTSB, normalized relative to the mean level of the RNA transcripts, or the corresponding expression product, in a cancer tissue reference set of at least 30 cancer tissue specimens or the product of two or more reference normalizing genes, indicates that said treatment is not likely to provide a benefit to the patient, and (b) over-expression of the transcript of one or more of Hepsin, ErbB3, MTA, Gus, and VEGF, normalized relative to the mean level of the RNA transcripts, or the corresponding expression product, in a cancer tissue reference set of at least 30 cancer tissue specimens or the product of two or more reference normalizing genes, indicates that said treatment is likely to provide a benefit to the patient.

3. The method of claim 2 comprising determining the expression level of at least 2 of said prognostic transcripts or their expression products.

4. The method of claim 2 comprising determining the expression level of at least 5 of said prognostic transcripts or their expression products.

5. The method of claim 1 wherein over-expression is determined relative to the mean level of the RNA transcript or the product of two or more reference normalizing genes.

6. The method of claim 1 wherein said cancer cells comprise a cancer selected from the group consisting of ovarian cancer, colon cancer, pancreatic cancer, non-small cell lung cancer, breast cancer, and head and neck cancer.

7. The method of claim 1 where the cancer cells are fixed, paraffin-embedded, or fresh, or frozen.

8. The method of claim 1 where the cancer cells are from fine needle, core, or other types of biopsy.

9. The method of claim 1 wherein the cancer cells are is obtained by fine needle aspiration, bronchial lavage, or transbronchial biopsy.

10. The method of claim 1 wherein the expression level of said prognostic RNA transcript or transcripts is determined by reverse transcriptase-PCR.

11. The method of claim 1 wherein the expression level of said expression product is determined by immunohistochemistry.

12. The method of claim 1 wherein the expression level of said expression product is determined by proteomics technology.

13. The method of claim 1 wherein the method for measurement of the prognostic RNA transcript or its expression product is provided in the form of a kit or kits.

14. The method of claim 1 wherein the EGFR inhibitor is an antibody or an antibody fragment.

15. The method of claim 1 wherein the EGFR inhibitor is a small molecule.

16. An array comprising polynucleotides hybridizing to the following genes: STAT5A, STAT5B, WISP1, CKAP4, FGFr1, cdc25A, RASSF1, G-Catenin, H2AFZ, NME1, NRG1, BC12, TAGLN, YB1, Src, IGF1R, CD44, DIABLO, TIMP2, PDGFrA, CTSB, Hepsin, ErbB3, MTA, Gus, and VEGF, immobilized on a solid surface.

17. An array comprising polynucleotides hybridizing to the following genes: STAT5A, STAT5B, WISP1, CKAP4, FGFR1, cdc25A, RASSF1, G-Catenin, H2AFZ, NME1, NRG1, BC12, TAGLN, YB1, Src, IGF1R, CD44, DIABLO, TIMP2, PDGFRa, and CTSB.

18. An array comprising polynucleotides hybridizing to the following genes: Hepsin, ErbB3, MTA, Gus, and VEGF.

19. The array of claim 16 wherein said polynucleotides are cDNAs.

20. The array of claim 19 wherein said cDNAs are about 500 to 5000 bases long.

21. The array of claim 16 wherein said polynucleotides are oligonucleotides.

22. The array of claim 21 wherein said oligonucleotides are about 20 to 80 bases long.

23. The array of claim 22 which comprises about 330,000 oligonucleotides.

24. The array of claim 16 wherein said solid surface is glass.

25. A method of preparing a personalized genomics profile for a patient, comprising the steps of;
(a) subjecting RNA extracted from cancer cells obtained from the patient to gene expression analysis;
(b) determining the expression level in the cancer cells of STAT5B, normalized relative to the mean level of the RNA transcripts in a cancer tissue reference set of at least 30 cancer tissue specimens or the product of two or more reference normalizing genes; and
(c) creating a report summarizing the data obtained by said gene expression analysis.

26. The method of claim 25 wherein said cancer cells are obtained from a fixed, paraffin-embedded biopsy sample.

27. The method of claim 26 wherein said RNA is fragmented.

28. The method of claim 25 wherein said report includes prediction of the likelihood that the patient will respond to treatment with an EGFR inhibitor.

29. The method of claim 30 wherein the cancer cells are lung cancer.

30. The method of claim 25 wherein the cancer cells comprises a cancer selected from the group consisting of colon cancer, head and neck cancer, lung cancer and breast cancer.

31. The method of claim 25 wherein said report includes recommendation for the best treatment option available to the patient.

32. A method for amplification of a gene selected from the group consisting of STAT5A, STAT5B, WISP1, CKAP4, FGFr1, cdc25A, RASSF1, G-Catenin, H2AFZ, NME1, NRG1, BC12, TAGLN, YB1, Src, IGF1R, CD44, DIABLO, TIMP2, PDGFRA, CTSB, Hepsin, ErbB3, MTA, Gus, and VEGF by polymerase chain reaction (PCR), comprising performing said PCR by using a corresponding amplicon listed in Table 3, and a corresponding primer-probe set listed in Table 4.

33. A PCR primer-probe set listed in Table 4.

34. A PCR amplicon listed in Table 3.

35. A prognostic method comprising:
(a) subjecting a sample comprising cancer cells obtained from a patient to quantitative analysis of the expression level of the RNA transcript of STAT5B, or its products, and
(b) identifying the patient as not likely to benefit from treatment with an EGFR inhibitor if the expression levels of said STAT5B RNA transcript or its products normalized relative to the mean level of the RNA transcripts or their products in a cancer tissue reference set of at least 30 cancer tissue specimens or the product of two or more reference normalizing genes, are elevated above a defined expression threshold.

36. The method of claim 35 wherein said cancer cells are selected from the group consisting of colon cancer, head and neck cancer, lung cancer and breast cancer cells.

37. The method of claim 35 comprising:
(a) subjecting a sample comprising cancer cells obtained from a patient to quantitative analysis of the expression level of the RNA transcript of at least one gene selected from the group consisting of Hepsin, ErbB3, MTA, Gus, and VEGF, or their product, and
(b) identifying the patient as likely to benefit from treatment with an EGFR inhibitor if the expression levels of said RNA transcripts or their products, normalized relative to the mean level of the RNA transcripts in a cancer tissue reference set of at least 30 cancer tissue specimens or the product of two or more reference normalizing genes, are elevated above a defined expression threshold.

38. The method of claim 37 wherein said cancer cells are selected from the group consisting of non-small cell lung cancer (NSCLC) cells, colon cancer cells, head and neck cancer cells, lung cancer cells and breast cancer cells.

39. The method of claims 35 wherein the reference normalizing genes are selected from the group consisting of glyceraldehyde-3-phosphate dehydrogenase (GAPDH), Cyp1, albumin, actins, tubulins, cyclophilin hypoxantine phosphoribosyltransferase (HRPT), L32, 28S, and 18S.

40. The method of claim 35 wherein the sample is subjected to global gene expression analysis of all genes present above the limit of detection.

41. The method of claim 40 wherein the levels of the RNA transcripts of said genes are normalized relative to the mean level of the RNA transcripts in a cancer tissue reference set of at least 30 cancer tissue specimens.

42. The method of claim 41 wherein the level of RNA transcripts is determined by quantitative RT-PCR (qRT-PCR), and the signal is a Ct value.

43. The method of claim 42 wherein the assayed genes include at least 50 cancer related genes.

44. The method of claim 42 wherein the assayed genes includes at least 100 cancer related genes.

45. The method of claim 35 wherein said patient is human.

46. The method of claim 45 wherein said cancer cells are in a fixed, paraffin-embedded tissue (FPET) sample, or fresh or frozen tissue sample.

47. The method of claim 45 wherein cancer cells are a tissue sample from fine needle, core, or other types of biopsy.

48. The method of claim 45 wherein said quantitative analysis is performed by qRT-PCR.

49. The method of claim 45 wherein said quantitative analysis is performed by quantifying the products of said genes.

50. The method of claim 49 wherein said products are quantified by immunohistochemistry or by proteomics technology.

51. The method of claim 35 further comprising the step of preparing a report indicating that the patient has a decreased likelihood of benefit from treatment with an EGFR inhibitor.

52. A kit comprising one or more of (1) extraction buffer/reagents and protocol; (2) reverse transcription buffer/reagents and protocol; and (3) qPCR buffer/reagents and protocol suitable for performing the method of claim 1.

53. The method of claim 35 comprising:
(a) subjecting a sample comprising cancer cells obtained from a patient to quantitative analysis of the expression level of the RNA transcript of at least one gene selected from the group consisting of STAT5A, WISP1, CKAP4, FGFR1, cdc25A, RASSF1, G-Catenin, H2AFZ, NME1, NRG1, BC12, TAGLN, YB1, Src, IGF1R, CD44, DIABLO, TIMP2, PDGFRa, and CTSB, or their products, and
(b) identifying the patient as not likely to have a benefit from treatment with an EGFR inhibitor if the expression levels of said RNA transcripts normalized relative to the mean level of the RNA transcripts in a cancer tissue reference set of at least 30 cancer tissue specimens or the product of two or more reference normalizing genes, or their products, are elevated above a defined expression threshold.
